(19) United States
(12) Patent Application Publication
(10) Pub. No.: US 2012/0226216 A1
(43) Pub. Date: Sep. 6, 2012

(54) APPLICATOR FOR INSERTING AN ELONGATED OBJECT INTO A HUMAN OR ANIMAL BODY ORIFICE

(76) Inventors: David Conroy, New York, NY (US); Axel Hammen, Lengnau (CH)

(21) Appl. No.: 13/499,988

(22) PCT Filed: Oct. 13, 2010
(86) PCT No.: PCT/CH2010/000255

§ 371 Date: May 21, 2012

(30) Foreign Application Priority Data
Oct. 14, 2009 (CH) 1578/09

(51) Int. Cl. A61F 13/26; A61F 13/34

(52) U.S. Cl. 604/11

(57) ABSTRACT

The invention relates to an applicator (1) for inserting an elongated object into a human or animal body orifice, comprising a one-piece, elongated applicator body (2). An accommodating area (5) for the object is arranged at a first end (3), and a handle area (6) is arranged at the second area (4). The accommodating area (5) is hollow and is open at the first end (3). At least one slit (9) open at the first end (3) extends from said end (3) in the wall of the accommodating area (5) in order to accommodate a flexible retracting means arranged on an object to be inserted by means of the applicator.

The invention relates to an applicator for inserting an elongated object into a human or animal body orifice, consisting of a one-piece elongated applicator body having a first end with a receiving section for the object and a second end with a handle portion, the receiving section being hollow and open at the first end in order to enclose the object on part of its length, and at least one stop surface being provided in the receiving section in order to limit the insertion depth of the object in the receiving section during the insertion of the object into the applicator and to apply an axial force to the object during the introduction of the object into the body orifice.

Applicators of this kind, which do not require an ejector that is axially displaceable in the applicator body, are e.g. used for inserting tampons for women's menstrual hygiene but may also be used for inserting diagnostic or therapeutic apparatus. Their operation is based upon the fact that the friction between the object and the body tissue is greater than the friction between the object and the receiving section of the applicator so that the object is separated from the applicator and remains in the body orifice when the applicator is withdrawn after the insertion.

Patent EP800375B1 describes an applicator of this kind that is intended to be sold together with so-called digital tampons, the user having the choice of using the tampons with or without the help of the applicator.

Tampons as well as the aforementioned diagnostic or therapeutic apparatus are usually provided at their rear ends as seen in the insertion direction with a flexible pulling means, for example a string, for withdrawing the tampon or the object, respectively. In the case of the diagnostic or therapeutic apparatus, the pulling means may also contain conductors for the transmission of energy and/or signals.

In tampon applicators that are furnished with the tampon inserted in the applicator, the withdrawal string is usually led through the interior of the applicator and projects at the rear end thereof. The reference U.S. Pat. No. 2,355,628 shows such a tampon and applicator combination. It is obvious that it would be difficult for a person wishing to insert a tampon into such an applicator by herself to thread the withdrawal string into the applicator as shown in the reference.

Regarding the aforementioned applicator according to EP800375B1, a tampon having a withdrawal string is depicted, see e.g. FIG. 3 or 9, the string being arranged at the rear end of the tampon in such a manner that its free end hardly projects from the end of the tampon. It is therefore not ensured that the end of the string will project from the body orifice after the insertion of the tampon and can be gripped without problems to withdraw the tampon.

It is conceivable to insert a tampon having a withdrawal string into an applicator of the type depicted in EP800375B1 in such a manner that the string is deflected around the forward edge of the receiving section and guided backwards so that an end of the string will always project from the body. However, this would entail the disadvantage that the string would have to be substantially longer than if it were guided through the interior of the applicator. In the case of a relatively thick and/or stiff withdrawal means, e.g. one accommodating conductors, as mentioned above, a deflection around the forward edge of the applicator would be difficult and a thus deflected withdrawal means would disturb during the insertion of the object.

On the background of this prior art, the invention is based upon the object of suggesting an applicator that is so designed that the person inserting the object can insert the object into the applicator by himself or herself and that during and after the insertion of the object into a body orifice, an end of the withdrawal means remains outside the body. Another object of the invention is to suggest an applicator that is suitable for inserting an object having a relatively thick and/or stiff withdrawal means. Another object of the invention is to suggest an applicator that is suitable for inserting an object having a relatively short withdrawal means. Ultimately, another object of the invention is to provide an applicator that is reusable.

According to the invention, these objects are attained in that at least one slot that is open at the first end extends from the first end in the wall of the receiving section in the longitudinal direction of the applicator body.

This solution offers the advantage that an object to be inserted into a body orifice by means of the applicator can be inserted in the receiving section of the applicator in such a manner that a withdrawal means fastened to the rear end of the object is placed in the slot so that an end of the withdrawal means always remains outside the body orifice. Due to the guidance of the withdrawal means in the slot, the external circumference of the receiving section remains free so that the withdrawal means cannot disturb during the insertion.

According to one embodiment, the slot extends in the longitudinal direction of the applicator body past the stop surface. In this manner it is ensured that a flexible withdrawal means fastened to the rear end of an object inserted into the applicator 1 can be guided to the outside of the applicator without an excessive deflection.

According to further embodiments, the slot has a constant width over its length or a width that increases from the first end. In this manner it is prevented that body tissue may be caught in the open end of the slot during the insertion of the applicator into a body orifice and subsequently gripped in the slot during the further insertion of the applicator.

According to another embodiment, the external diameter of the receiving section increases from the first end toward the second end up to a maximum that is preferably located between the first end and the stop surface. This measure causes the opening edge of the receiving section 5 to lie against the lateral surface of an accordingly dimensioned object received therein and to retain the latter in the receiving section. Additionally, this design prevents that the opening edge of the receiving section may disturb during the insertion of the applicator.

Another embodiment is characterized in that the applicator body is tapered from the receiving section toward the second end. A so designed applicator is easy to handle and easy to withdraw after its insertion. In addition, a withdrawal means may extend on the outside of the tapered area in every phase of the insertion and of the withdrawal without disturbing.

According to a further embodiment, the handle portion is formed by an enlargement at the second end of the applicator body. This enlargement is helpful during the insertion and the withdrawal of the applicator.

According to a further embodiment, the enlargement is provided with a recess that is open toward the outer edge of the enlargement. The recess allows receiving the flexible withdrawal means of an object inserted in the applicator so that it is retained during the insertion and does not hang loosely and disturb.

In another embodiment, the stop surface is located on at least one inwardly salient indentation in the wall of the applicator body. In this case, the indentation can be so designed that no so-called dirt corners will form and the applicator is thus easy to clean.

According to an alternative embodiment, the stop surface is an annular surface on the inside of a circumferential step in the wall of the applicator body. This applicator body is simple to manufacture and also offers the advantage of easy and thorough cleaning when the edges of the steps are correspondingly rounded.

According to another alternative embodiment, the stop surface is formed on a longitudinal ridge that projects inwards from the wall of the applicator body.

Preferentially, multiple longitudinal ridges are arranged on the inner circumference of the applicator body. With regard to mold technology, the manufacture of this applicator is relatively simple.

According to yet another embodiment, the applicator body is hollow over its entire length and the slot extends over the entire length of the applicator body. This embodiment combines the advantages of the easy insertion of a flexible withdrawal means and of the central guidance of this withdrawal means in the applicator body.

Ultimately, according to another embodiment, the applicator body is made of synthetic material and is preferably injection-molded. In this manner, the applicator is easy to clean and reusable.

An exemplary embodiment of the invention will be explained in more detail hereinafter with reference to the appended drawings showing:

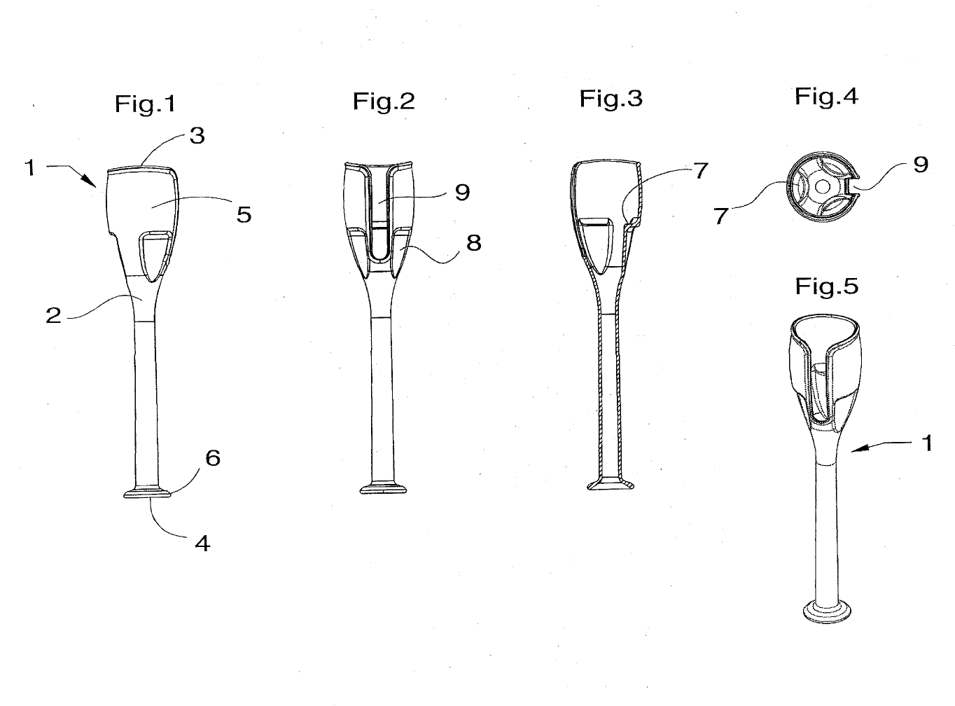

FIG. 1 a lateral view of a first exemplary embodiment of an applicator according to the invention,

FIG. 2 a lateral view of the same applicator rotated 90 degrees with respect to FIG. 1,

FIG. 3 a longitudinal section of the first exemplary embodiment,

FIG. 4 a top view of the first exemplary embodiment,

FIG. 5 a perspective view of the first exemplary embodiment,

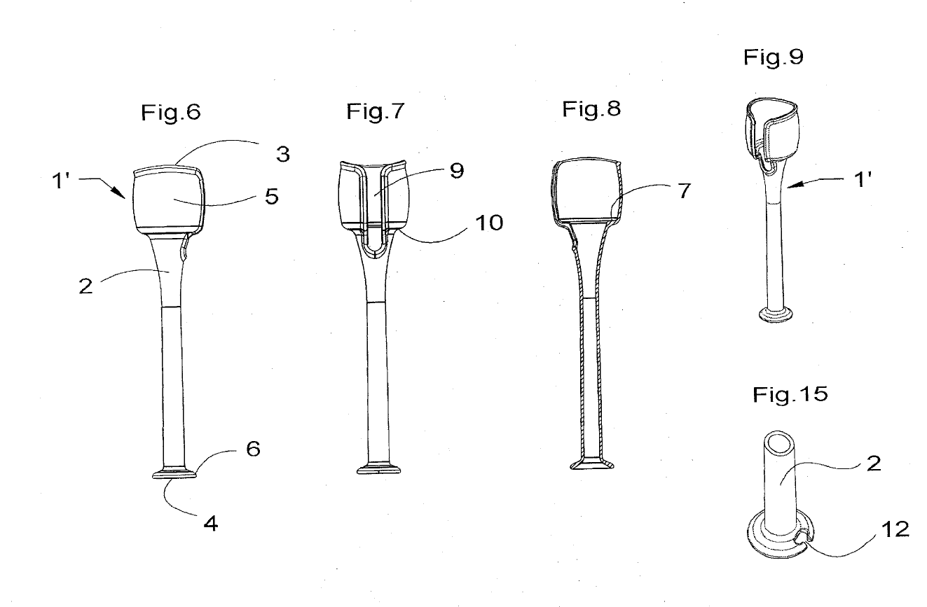

FIG. 6 a lateral view of a second exemplary embodiment of an applicator according to the invention,

FIG. 7 a lateral view of the applicator according to FIG. 6 rotated 90 degrees with respect to FIG. 6,

FIG. 8 a longitudinal section of the second exemplary embodiment,

FIG. 9 a perspective view of the applicator according to FIGS. 6 to 8,

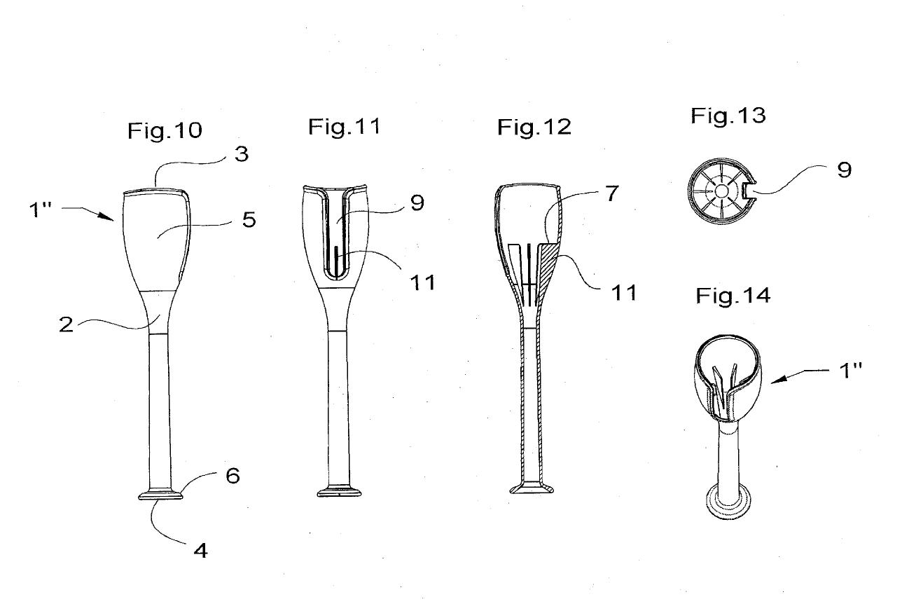

FIG. 10 a lateral view of a third exemplary embodiment of an applicator according to the invention,

FIG. 11 a lateral view of the applicator according to

FIG. 10 rotated 90 degrees with respect to FIG. 10,

FIG. 12 a longitudinal section of the third exemplary embodiment,

FIG. 13 a top view of the third exemplary embodiment,

FIG. 14 a perspective view of the third exemplary embodiment,

FIG. 15 a particular embodiment of the handle portion that can be implemented in all exemplary embodiments of the applicator.

In all drawing figures described hereinafter, the same reference numerals are used for the same parts. FIGS. 1 to 5 show a first exemplary embodiment of an applicator 1 according to the invention in different views. Applicator 1 consists of an elongated applicator body 2 that is manufactured in one piece, e.g. injection-molded from synthetic material. Applicator body 2 has a first end 3 that is located at the top in FIGS. 1 to 3 and 5. On its opposite side, applicator body 2 has a second end 4. At its first end 3 begins a receiving section 5 for an object to be inserted into a body orifice by means of the applicator, e.g. a tampon for women's menstrual hygiene. Receiving section 5 extends from first end 3 to three stop surfaces 7. The latter are formed at the upper ends of indentations 8 that are arranged at angular distances of 120 degrees and extend inwardly from the wall of applicator body 2. As appears in the figures, the wall of the receiving section does not have the shape of a hollow cylinder but is slightly cambered. Therefore, the opening edge of the receiving section is slightly turned inwards and thus less disturbing during the insertion. In the wall of the applicator body a slot 9 is provided which is open at first end 3 and extends in the longitudinal direction of applicator 1 past stop surfaces 7, as seen particularly clearly in FIG. 2. At second end 4, applicator body 2 is enlarged and forms a handle portion 6 that is helpful particularly for withdrawing the applicator after the insertion of an object.

In FIGS. 6 to 9, a second exemplary embodiment 1′ of the applicator according to the invention is illustrated. It is clearly visible that the outer contour of this applicator 1′ differs from the outer contour of the applicator of FIGS. 1 to 5. In the area designated by reference numeral 10, the applicator body is stepped so that an annular stop surface 7 is formed on the inside.

FIGS. 10 to 14 show a third exemplary embodiment 1″ of the applicator. Here, the outer contour of the applicator 1″ corresponds to that of the example according to FIGS. 1 to 5. In this example, however, several longitudinal ridges 11 are provided which are arranged at the end of receiving section 5 and distributed on the inner circumference, and whose front surfaces facing towards the first end form the stop surfaces 7.

Ultimately, FIG. 15 shows a particular embodiment of handle portion 6 at the second end 4 of the applicator body in a broken perspective view. A recess 12 that is open at the edge of the handle portion allows receiving the withdrawal means of an object inserted in the applicator, e.g. the string of a tampon. Advantageously, recess 12 is inwardly enlarged as shown in the Figure so that a withdrawal means introduced therein will not easily fall out of recess 12.

The application of all described examples 1, 1′, and 1″ of the applicator is the same and will be described by way of example with reference to a commercially available tampon. A tampon is taken from its packaging and the withdrawal string is stretched so that it hangs loosely. Then the rearward end of the tampon, to which the withdrawal string is attached, is pushed into the receiving section 5 of the applicator up to stop surfaces 7 while simultaneously placing the withdrawal string in slot 9. If a recess according to FIG. 12 is provided in handle portion 6, the withdrawal string can be loosely inserted therein so that the string is guided along the outside of the applicator body and will not disturb during the subsequent insertion. Due to the slightly inwardly turned opening edge at the open end of receiving section 5, the tampon is retained in the applicator and can now be inserted into the vagina. As soon as the tampon is in place in the correct location, the applicator can be withdrawn while the tampon is retained in place by the elasticity of the vagina and the friction between the tampon and the surrounding mucosa. After the complete withdrawal of the applicator, the forwardly open slot 9 and—if provided—also the outwardly open recess 12 will immediately release the withdrawal string.

### LIST OF REFERENCE NUMERALS

1, 1′, 1″ applicator

2 body

3 first end

4 second end

5 receiving section

6 handle portion

7 stop surface

8 indentation

9 slot

10 constriction

11 ridge

12 recess

1. An Applicator for inserting an elongated object into a human or animal body orifice, comprising a one-piece elongated applicator body having a first end with a receiving section for the object and a second end with a handle portion, the receiving section being hollow and open at the first end in order to enclose the object on part of its length, and at least one stop surface being provided in the receiving section in order to limit the insertion depth of the object in the receiving section during the insertion of the object into the applicator and to apply an axial force to the object during the insertion of the object into the body orifice, wherein at least one slot that is open at the first end extends in the wall of the receiving section from the first end in the longitudinal direction of the applicator body.

2. The applicator according to claim 1, wherein the slot extends in the longitudinal direction of the applicator body past the stop surface.

3. The applicator according to claim 1, wherein the slot has a constant width over its length.

4. The applicator according to claim 1, wherein the slot has a width that increases over its length from the first end.

5. The applicator according to claim 1, wherein the external diameter of the receiving section increases from the first end toward the second end up to a maximum that is preferably located between the first end and the stop surface.

6. The applicator according to claim 1, wherein the applicator body is tapered from the receiving section toward the second end.

7. The applicator according claim 1, wherein the handle portion is formed by an enlargement at the second end of the applicator body.

8. The applicator according to claim 7, wherein the enlargement is provided with a recess that is open toward the outer edge of the enlargement.

9. The applicator according to claim 1, wherein the stop surface is located on at least one inwardly salient indentation in the wall of the applicator body.

10. The applicator according to claim 1, wherein the stop surface is an annular surface on the inside of a circumferential step in the wall of the applicator body.

11. The applicator according to claim 1, wherein the stop surface is formed on a longitudinal ridge that projects inwards from the wall of the applicator body.

12. The applicator according to claim 1, wherein the applicator body is hollow over its entire length and the slot extends over the entire length of the applicator body.

13. The applicator according to claim 1, wherein the applicator body is made of synthetic material and is preferably injection-molded.

14. An Elongated object for insertion into a human or animal body orifice, including a tampon and a therapeutic object, the elongated object being provided at its rear end, as seen in the direction of introduction, with flexible withdrawal means for withdrawing the elongated object.

15. The object according to claim 14, wherein the flexible withdrawal means contains conductors for the transmission of energy and/or signals.

16. An applicator assembly comprising an applicator according to claim 1 and an elongated object, more particularly including a tampon and/or a therapeutic apparatus, the elongated object being provided at its rear end with a flexible withdrawal means, and the elongated object being partly enclosed on its length by the applicator.

17. The applicator assembly according to claim 16, wherein the flexible withdrawal means is guided along the outside of the applicator body.

18. The applicator assembly according to claim 16, wherein the flexible withdrawal means contains conductors for the transmission of energy and/or signals.

19. The applicator assembly according to claim 16, wherein the elongated object is for insertion into a human or animal body orifice, including a tampon and a therapeutic object, the elongated object being provided at its rear end, as seen in the direction of introduction, with flexible withdrawal means for withdrawing the elongated object.

20. A method for using an applicator according to claim 1 for inserting an elongated object, more particularly including a tampon and/or a therapeutic apparatus, into a human or animal body orifice, wherein the elongated object is pushed by its rear end, to which a withdrawal means is fastened, into the receiving section of the applicator up to stop surfaces thereof, so as to be enclosed on part of its length, wherein the withdrawal means is simultaneously loosely placed in the slot so that the withdrawal means is guided along the outside of the applicator body and does not disturb during the subsequent insertion, wherein the elongated object is being retained in the applicator by a slightly inwardly turned opening edge at the open end of the receiving section for being inserted into the body orifice and wherein, as soon as the elongated object is in place at the correct location in the body orifice, the applicator is withdrawn while the elongated object is retained in place by the elasticity of the body orifice and the friction between the elongated object and the surrounding mucosa.

21. The method according to claim 20, wherein the elongated object is for insertion into a human or animal body orifice, including a tampon and a therapeutic object, the elongated object being provided at its rear end, as seen in the direction of introduction, with flexible withdrawal means for withdrawing the elongated object.
